(19) United States
(12) Patent Application Publication
(10) Pub. No.: US 2015/0190111 A1
(43) Pub. Date: Jul. 9, 2015

(54) ULTRASOUND-GUIDED NON-INVASIVE BLOOD PRESSURE MEASUREMENT APPARATUS AND METHODS

(71) Applicant: William R. Fry, Lexington, SC (US)

(72) Inventor: William R. Fry, Lexington, SC (US)

(21) Appl. No.: 14/585,717

(22) Filed: Dec. 30, 2014

Related U.S. Application Data
(60) Provisional application No. 61/923,335, filed on Jan. 3, 2014.

(51) Int. Cl. A61B 8/04; A61B 8/00

(52) U.S. Cl. CPC A61B 8/04; A61B 8/461; A61B 8/4472; A61B 8/429; A61B 8/56; A61B 8/4433; A61B 8/4427

(57) ABSTRACT

A non-invasive blood (or compartment) pressure measurement device comprises a hand-held housing configured to fit over, or couple to, an existing ultrasound probe. At least one force sensor in or on the housing generates a signal representing the amount of force applied by the probe as a user manipulates the housing. Electronic circuitry converts the force signal into an estimate of pressure when the display shows that a particular vessel or compartment has been occluded by the force of the probe. Apparatus may be provided to grip the handle portion of the probe, with at least one force sensor is supported on or in a component disposed between the apparatus and the housing. The component may be a thin-walled tube, and a plurality of strain gages, each forming a Wheatstone bridge load cell, may be disposed circumferentially around the component. The electrically circuitry is further operative to sum the signals from the plurality of strain gages to reject non-axial moments.

### REFERENCE TO RELATED APPLICATION

This application claims priority from U.S. Provisional Patent Application Ser. No. 61/923,335, filed Jan. 3, 2014, the entire content of which is incorporated herein by reference.

### FIELD OF THE INVENTION

This invention relates generally to non-invasive blood pressure measurement and, in particular, to ultrasound-guided apparatus and methods that do not require modification to existing ultrasound probes.

### BACKGROUND OF THE INVENTION

Measuring the pressure in a blood vessel, extremity compartment, or the abdomen is a routine part of patient care. “Blood pressure” most commonly refers to arterial pressure, which commonly measured indirectly in healthy people. The method involves placing a pressurized cuff around the arm to detect the force of blood pulsing through the arm's arteries. The measurement represents how hard the blood is being forced out by the left ventricle (systolic pressure), and how the ventricles are preparing for the next contraction (diastolic pressure).

Whereas arterial blood pressure is a measure of the pressure of the blood leaving the heart, the pressure entering the heart may be of critical importance. All venous blood drains into the right atrium, and the pressure in the vena cava just outside the right atrium is called the central venous pressure (CVP). CVP provides an indication of the volume of blood that is flowing through the veins and into the right atrium. With a failing heart blood tends to “back up” in the veins, causing CVP to increase. Thus, CVP measurement may be an important tool in diagnosing potential heart failure and other conditions associated with low cardiac output.

It is challenging to obtain a quick and accurate measurement of CVP. Current methods include either invasive monitoring with catheters or noninvasive estimates that give a very broad range of values that are of limited value due to their lack of specificity. Invasive methods involve threading a catheter along a major vein until it is within the vicinity of the right atrium. Pressure readings are then collected directly from inside the vein. However, threading a central line is time-consuming, difficult to perform, and risky. Needle insertion can result in internal bleeding if an artery is accidentally punctured, inadvertent collapse of the lung, and risk of infection is also present whenever an artificial device in placed in the body.

To avoid initial catheterization, CVP may be estimated by treating the superior vena cava as a manometer to the right atrium. The pressure at the right atrium correlates to the height of the column of blood in the vein, which can be estimated by visually identifying small disturbances in the jugular vein. However, this method is prone to error, and the process of physically positioning a patient for the procedure is not well-suited to emergency situations.

With the advent of bedside ultrasound, clinicians now use ultrasound routinely in patient assessment. By being able to measure the applied pressure placed on the ultrasound probe, arterial, venous, and compartment pressures can be measured under direct visualization, enabling more accurate data to be obtained for patient care decisions at the time of measurement or for trending.

It is known to use ultrasound to visualize cardiovascular structures to aid in CVP measurement. Published U.S. Patent Application No. 2007/0239041 describes apparatus and methods for non-invasive measurement of a subject's venous pressure, including CVP. The method uses an ultrasound system to visualize the internal jugular (IJ) vein. The apparatus further comprises a probe with a load cell. Once the IJ has been located, the operator pushes on the surface of the neck with the probe until the external pressure is sufficient to collapse the IJ. The load cell within the probe determines the amount of force applied, and the applied force is converted into venous pressure. While this approach does not require any alteration to the ultrasound probe, it does require a separate piece of equipment and both hands for the required manipulation.

If a force sensor could be positioned between the tip of the probe and the skin of the subject, blood pressure measurements could be carried out using only the pressure applied by the probe itself. One known technique uses an ultrasound probe modified with a quartz pressure transducer within a mixture of water and glycerin that is translucent to ultrasound waves. The device records the external pressure needed to collapse the IJ and correlates this value to the CVP. However, this solution requires modification of the ultrasound probe, which makes it unattractive for clinicians wishing to use existing ultrasound equipment.

Another approach, described in Published U.S. Patent Application No. 2011/0137173 resides in a combined blood flow and pressure measurement device for hemodynamic monitoring that includes a Doppler ultrasound probe combined with arterial pressure measurement, or a signal input from a suitable pressure transducer system. The blood pressure data is obtained from an external source pressure transducer derived from pressure measurements made through invasive monitoring from arterial lines, pulmonary artery pressure catheters, central venous lines, etc. The system couples the Doppler data obtained from ultrasound with the pressure data and displays both on a monitor.

### SUMMARY OF THE INVENTION

This invention improves upon the prior art by providing a hand-held, force-sensing instrument that operates in conjunction with an existing ultrasound probe. Pressure is applied to the skin of a patient through the hand-held unit by a user of the ultrasound probe. When the ultrasound image indicates that a particular vessel or compartment has been sufficiently compressed, occluded or deformed, the force applied to the probe is converted into a pressure reading and displayed.

The invention enables non-invasive arterial, venous, or compartment pressure measurement, and existing ultrasound equipment may be used without modification. If a cine loop is generated by the equipment, the scrolling back of the images to look at coaptation, vessel wall deformation, or deformation of the fascia can be reviewed with the corresponding pressure obtained at that instant. This may be done through a wireless transmission of pressure data to the ultrasound machine and software additions to allow the pressure data to be displayed on the ultrasound screen in real time. Alternatively, a display of pressure on the device itself can be used to visually see the pressure exerted. A button on the device can be pushed to freeze the pressure gauge at that measurement. A series of pressures can be reviewed on the screen. Software averaging of pressures and deleting previous measurements completely or discarding a selected measurement.

A non-invasive blood (or compartment) pressure measurement device constructed in accordance with the invention comprises a hand-held housing configured to fit over, or couple to, an existing ultrasound probe. At least one force sensor in or on the housing generates a signal representing the amount of force applied by the probe as a user manipulates the housing. Electronic circuitry converts the force signal into an estimate of pressure when the display shows that a particular vessel or compartment has been occluded or deformed by the force of the probe. In all embodiments the blood vessel may be a vein, artery or compartment, such that “vessel” as used herein may refer to any of these.

Probes applicable to the invention include a handle portion transitioning to a wider head portion. In one embodiment of the invention, apparatus is provided to grip the handle portion of the probe. At least one force sensor is supported on or in a component disposed between the apparatus and the housing. In accordance with a preferred embodiment, the component is a thin-walled tube and the force sensor is a strain gauge.

In more preferred embodiments, a plurality of strain gages, each forming a Wheatstone bridge, are disposed circumferentially around the component. The circuitry is further operative to sum the signals from the plurality of strain gages to reject non-axial moments. The device may include a numerical readout for displaying the estimate of blood pressure, which is preferably presented in mm/Hg.

The system may further include a wireless transmitter for transmitting the blood pressure to a remote computer. Electronic circuitry and computer software may be provided enabling the estimate of blood pressure to be displayed on the display to which the ultrasound probe is coupled.

The device may include a rechargeable battery disposed in the housing, and a charging stand may be provided for receiving the housing for recharging purposes. The force sensor(s), electronic circuitry and other sensitive components within the housing may be coated or encapsulated to resist ultrasound coupling gel.

The housing may comprise a clamshell with an upper opening to receive the cord of the ultrasound probe. As an alternative to a probe handle grip, the housing may feature a lower edge with one or more force sensors that bear against the head portion of the probe during use.

A method of measuring blood or compartment pressure in accordance with the invention comprises the steps of providing an ultrasound probe coupled to a display showing an internal region of a body being compressed by the probe, and providing a hand-held device configured to fit over, or couple to, the ultrasound probe. The device includes a sensor and electronics to measure the amount of force applied by the probe so as to generate a pressure measurement when the display indicates that a blood vessel or compartment has been occluded or deformed by the applied force of the ultrasound probe.

### BRIEF DESCRIPTION OF THE DRAWINGS

FIG. 1A is a drawing of a preferred embodiment of the invention;

FIG. 1B shows the embodiment of FIG. 1A in an open configuration;

FIG. 2 depicts details regarding the force sensing and processing electronics;

FIG. 3 shows additional circuit details, including signal filtering;

FIG. 4 illustrates the microprocessor and Bluetooth radio;

FIG. 5 illustrates power supply circuits;

FIG. 6A shows an alternative embodiment of the invention including a charging stand;

FIG. 6B shows the instrument of FIG. 6A in use; and

FIG. 7 illustrates an alternative design using deformable extensions and strain gages.

### DETAILED DESCRIPTION OF THE INVENTION

This invention resides in a blood pressure measurement instrument that couples to an existing ultrasound probe. The instrument measures the force applied to the probe as the compression of a blood vessel is monitored. The instrument continuously measures the force of the ultrasound probe as it is pressed against the body, converting the force into units of blood pressure such as mm/Hg.

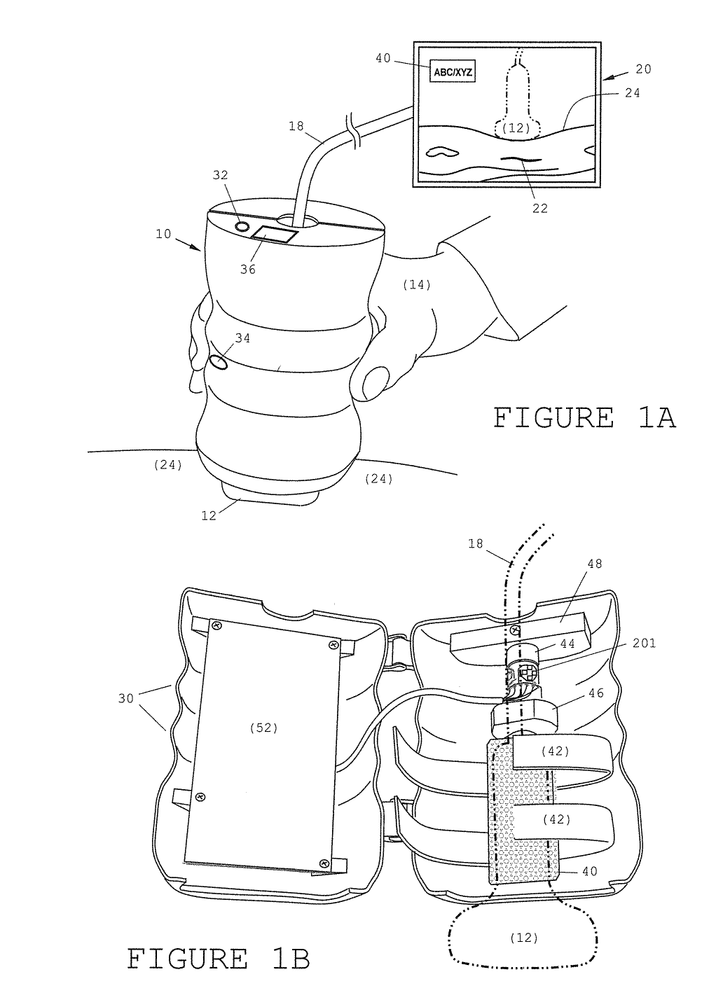

FIGS. 1A, 1B are drawings of a preferred embodiment of the invention. The instrument is depicted at 10, the probe at 12, and a user's hand at 14. The probe 12 interfaces to a monitor 20 through cord 18. The display 36 shows a blood vessel 24 collapsed though pressure applied by probe 12 onto the skin surface 24 of a patient.

The case of the instrument 10 preferably includes a plurality of ripples 30 or other features to enhance gripping. The case of the instrument also includes user controls such as “Zero” button 32, “Record” button 34 and pressure display 36. In addition to, or instead of display 36, the pressure reading may be wired or wirelessly transmitted to a monitor, including monitor 20 (numerical readout 40).

The case of the instrument in FIG. 1A is a clamshell design, which is shown open in FIG. 1B. The probe 12 is coupled to a non-skid pad 40 with hook-loop straps 42 to ensure that the instrument and probe move as a unit as pressure is applied. Pad 40 is, in turn, coupled to hollow tube 44 disposed between blocks 46, 48. As the probe 12 is forced against a surface 24 as shown in FIG. 1A, one or more strain gages 201, attached to tube 44, measure the applied force. The output of the strain gauges 201 are transferred to printed circuit board 52 containing processing electronics described in further detail below.

In the preferred embodiment, the force sensor is implemented as a load cell using 4 strain gages placed at 90 degree intervals around metal tube 44. Tube 44, which may be aluminum, is preferably machined to provide very thin walls to increase sensitivity to measure the small forces involved with measurement. A plurality of strain gages positioned circumferentially around the tube are used to detect and subtract out non-axial (moment) forces.

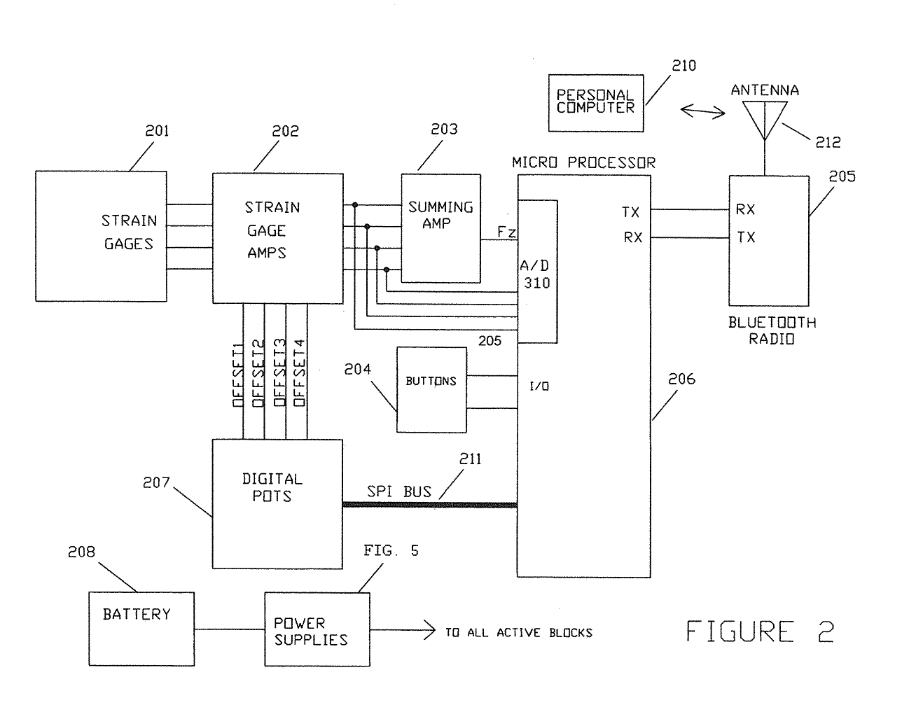

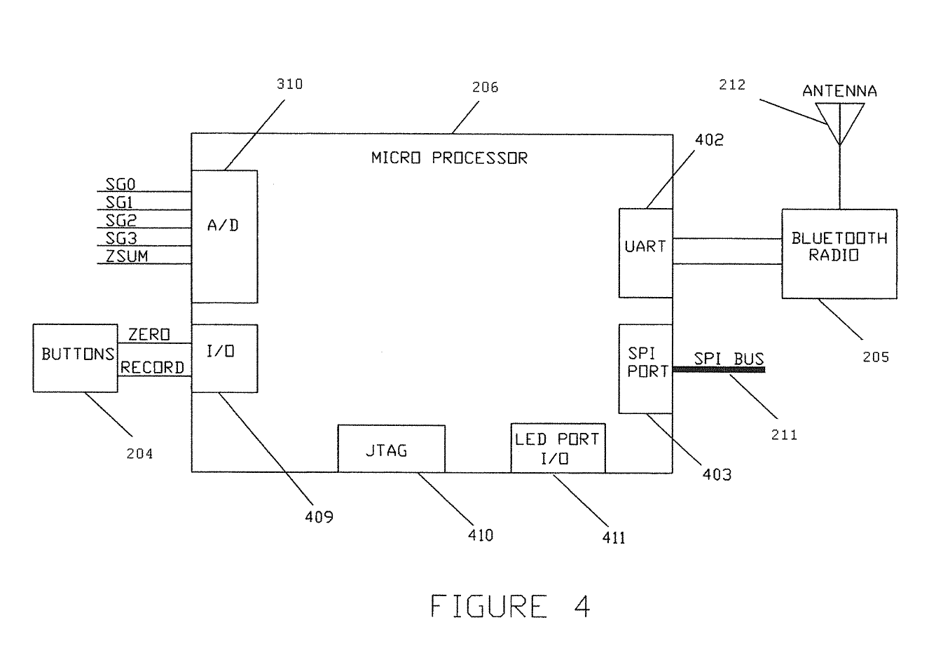

FIGS. 2-5 are block diagrams that illustrate the processing electronics. The four strain gages 201 are connected to four instrumentation amplifiers 202. The four instrumentation amplifier outputs are summed algebraically at amplifier 302 to generate force output signal Fz. The summing is important in rejecting off-center-load conditions. If the load is purely axial, then all the strain gage channels will produce the same output; however, if a moment is applied, then the outputs from the instrumentation amps will be different. If the moment causes the voltage from the strain gage located at zero degrees to be larger than the voltage from the strain gage located at 180 degrees, the summing amp will automatically correct for this inaccuracy.

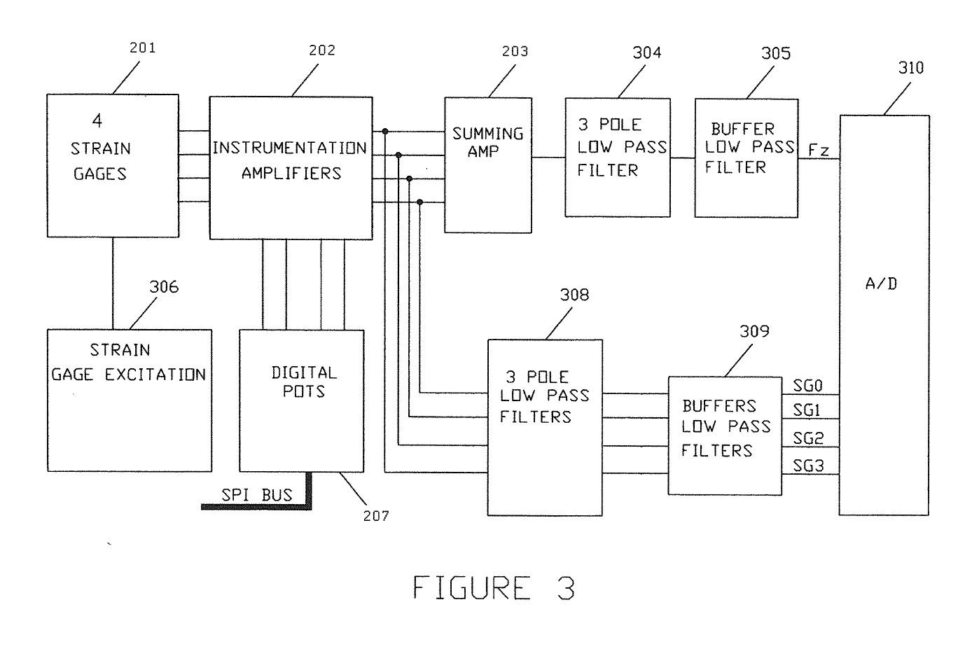

The output from the summing amp 203 is applied at to low-pass filter 304 shown in FIG. 3. The voltage from the low pass filter 304 applied to buffer 305 which amplifies and filters the signal filtered Z Sum signal (Fz) that connects to the A/D converter 310 located in microprocessor 206. Buffer 305 has a low-impedance output which is important, as the A/D convertor produces noise at its input during the A/D conversion. This low impedance signal helps reduce the noise level. The additional filtering also keeps high frequencies from entering the A/D convertor 310.

Microprocessor 206 is a very low-power device with seven 16-bit sigma-delta A/D convertors, a JTAG port 410 for programming and debugging, a 32 KHz crystal, a SPI port 211 for connection to the digital potentiometers (“pots”) 207 via SPI bus 211, and a UART 402 for connection to a Bluetooth radio 205 coupled to antenna 212. Microprocessor 206 also has I/O pins 409 that connect to the Zero and Record Buttons 204. Battery status and Bluetooth status indicators may also be provided.

In operation, when the Zero button is pressed, the microprocessor stores the Filtered_Fz voltage value in memory. When the ultrasound probe is pressed against the body, and the user wants to store this pressure value, the Record button is pressed and this voltage level is stored. The difference between the Zero value and the Record value is the pressure being applied to the body.

Digital pots 207 are used for offsetting the instrumentation amps 202 when no load is applied to the load cell. This is necessary because the strain gages are imperfect, and produce a small voltage when no strain is present. This small voltage is amplified hundreds of times by the instrumentation and buffer/summing amps 203. During calibration, the microprocessor executes a routine that sequentially drives the digital pots so that the voltage from the buffer amps is midway between an A/D reference voltage and analog ground. The values are then stored in the microprocessor's flash memory, and are loaded into the digital pots during start up.

The Bluetooth radio 205 connects to a PC (or any portable electronic devices including smartphones) via a small Bluetooth “dongle” that is connected to a USB port on the PC. The primary Bluetooth connections to the microprocessor are Tx (Transmit) , Rx (Receive), CT (Clear to Send), and RT (Request to Send). The TX and RX are standard UART signals with serial data bits. The CT and RT connections are used for flow control, which makes the serial communications more robust than using only TX and RX. The Bluetooth transceiver also has a Debug port for configuring the chip, which is accomplished using a Debug tool. A custom application program loaded into the PC communicates with the Bluetooth dongle and sends and receives data from the Bluetooth transceiver. The main functions of the custom application program are: Zero, Record, Display Pressure in mm/Hg.

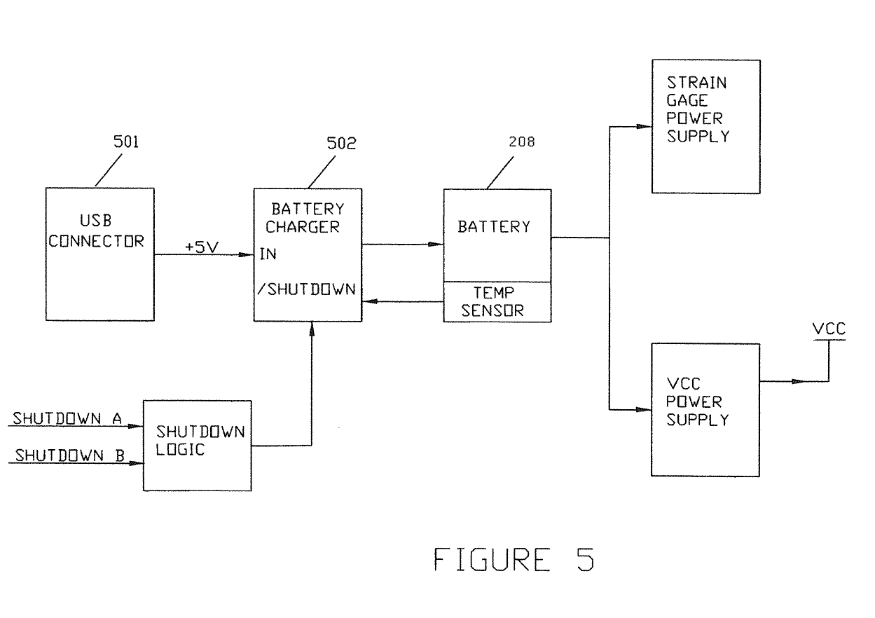

FIG. 5 illustrates power supply circuits. The electronics are powered by 2 Li-Polymer batteries 208 connected in parallel. The batteries are charged using an on-board charger 502. This chip gets power from a standard USB connector 501 which supplies 5V from some USB power such as a small wall mounted supply, or a PC. The batteries can be fully charged in about 3 hours. The batteries will stop charging when the batteries are fully charged. The supply 209 supplies +2.5V to block 306 connected to the strain gages for excitation.

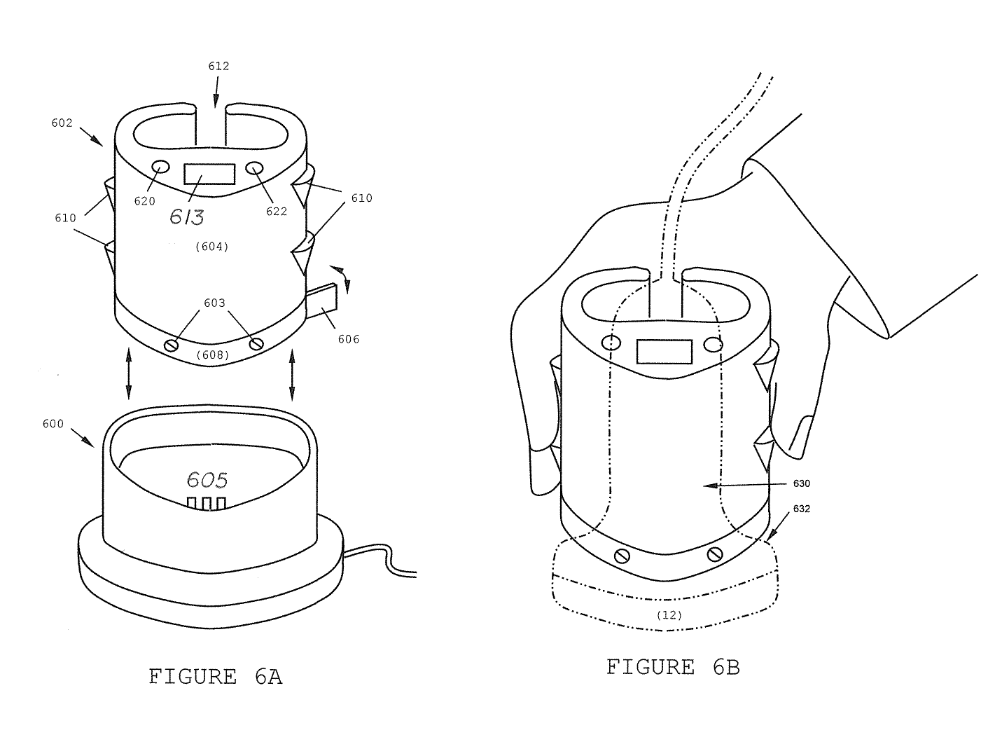

FIG. 6A shows an alternative embodiment of the invention including a charging stand 600, and FIG. 6B shows the instrument of FIG. 6A in use. The system includes a hand-held unit 602 received by a base unit 600 which includes contacts 605 that cooperate with corresponding contacts on the hand-held unit 602. The unit 602 contains a rechargeable battery as a power source, recharged through the base station which in turn is connected via cable 120 to AC power and/or a communications network as described herein.

Although the embodiment of FIG. 1 uses a Li-ion battery charged through a wired port, a charging stand of the type shown in FIG. 6A may also be used. In the embodiment of FIG. 6, the hand-held unit 602 includes an upper portion 604 and a lower portion 608. In contrast to gripping the handle 630 of the probe, one or more force sensors disposed between the lower portion of the housing and the head portion 632 of the probe which is larger than the bottom opening of the housing. As such, when pressure is applied to the lower portion of the housing through the upper portion, the electronics described herein converts the applied force signal from the force sensor(s) into a pressure measurement reading. As with other embodiment, the outer surface of the hand-held unit may include textures or features 610 to enhance gripping.

In this embodiment, the hand-held unit includes a gap 612 enabling the unit to be placed over an existing ultrasound probe without opening the case. The hand-held unit 602 is sized to attach to the probe 12 via friction; for example with an internal bore smaller that the distal flared end of a typical probe. Alternatively, an attachment mechanism such as screws 603 or an internal clamp operated by a lever 606 may be used to couple the hand-held unit to the probe body. The attachments of the device to the ultrasound probe, applicable to any of the embodiments disclosed herein, may be reusable but disposable.

The Zero and Record buttons are shown at 620, 622, respectively. As with other embodiments, attaching the unit to the ultrasound probe, and holding it up in the air via the device allows for zero balancing the system (i.e., probe plus device). The zero button cancels out the weight of the probe and/or the device. The pressure to deform or collapse the structure is acquired by either pushing the pressure acquisition (Record) button. An LCD display 613 will show the pressure measurement. Button functions to save or delete a reading may be activated through display prompting. A prompt to obtain consecutive readings may be averaged as an LCD screen prompt.

A barcode reader may be built into any of the instruments described herein to link the measurements to the proper patient through scanning the armband barcode. Low-energy Bluetooth or other wireless connections may be used to download readings directly to the patient's electronic medical record (EMR) or to a base station where it can then be downloaded to the EMR.

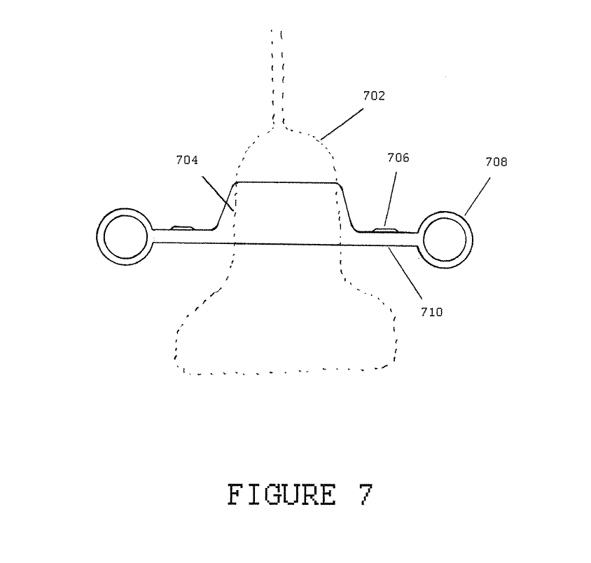

FIG. 7 illustrates a different physical configuration for the hand-held unit which uses deformable plastic arms 710 with optional finger loops 708 that bend slightly as pressure was applied to the patient. The ultrasound probe is depicted at 702. The attachment to the probe 702 may be through friction or an adhesive. By using two strain gages 706 (one on each “arm”), a correlation curve would be formed between the average amount of strain in the arms and the corresponding pressure being applied to the patient. This approach would have the advantage of being simple and inexpensive, as well as highly sensitive, since the strain gages are very sensitive to deformation in the material. One disadvantage might be the need to calibrate the attachment and take the average deformation between the two gages.

1. A non-invasive blood or compartment pressure measurement device adapted for use with an ultrasound probe in communication with a display, the device comprising:
a hand-held housing configured to fit over, or couple to, an existing ultrasound probe;
at least one force sensor outputting a signal representing the amount of force applied by the probe as a user manipulates the housing; and
electronic circuitry operative to convert the signal into pressure measurement when the display shows that the blood vessel or compartment has been occluded or deformed by the force of the probe.

2. The device of claim 1, wherein the ultrasound probe has a handle portion, and the device further comprises:
apparatus for gripping the handle portion of the probe;
a component disposed between the apparatus and the housing; and
at least one force sensor coupled to the component.

3. The device of claim 2, wherein the component disposed between the apparatus and the housing is a thin-walled tube; and
the force sensor is a strain gauge.

4. The device of claim 3, including a plurality of strain gages on the component forming a Wheatstone bridge load cell.

5. The device of claim 3, including a plurality of strain gages disposed circumferentially around the component; and
wherein the electrically circuitry is operative to sum the signals from the plurality of strain gages to reject non-axial moments.

6. The device of claim 1, wherein the measurement is blood pressure in mm/Hg.

7. The device of claim 1, further including a numerical readout for displaying the pressure measurement.

8. The device of claim 1, further including a wireless transmitter for transmitting the measurement to a remote computer.

9. The device of claim 1, further including electronic circuitry and computer software enabling the pressure measurement to be displayed on the display to which the ultrasound probe is coupled.

10. The device of claim 1, including a rechargeable battery disposed in the housing; and
a charging stand for receiving the housing for recharging purposes.

11. The device of claim 1, wherein the force sensor and electronic circuitry are coated or encapsulated to resist ultrasound coupling gel.

12. The device of claim 1, wherein the housing comprises a clamshell with an upper opening to receive the cord of the ultrasound probe.

13. The device of claim 1, wherein the ultrasound probe includes a head portion that is larger than the housing; and
the housing comprises a lower edge with one or more force sensors that bear against the head portion of the probe during use.

14. A method of measuring blood or compartment pressure, comprising the steps of:
providing an ultrasound probe coupled to a display showing a vessel or compartment being compressed or deformed by the probe;
providing a hand-held device configured to fit over, or couple to, the ultrasound probe, the device including a sensor and electronics to measure the amount of force applied by the probe during the compression or deformation; and
generating an pressure measurement when the display indicates that the vessel or compartment has been occluded or deformed by the applied force of the ultrasound probe.

15. The method of claim 14, including the step of providing the hand-held device of claim 1.

16. The method of claim 14, wherein the vessel is a vein or artery.

17. The method of claim 14, including the steps of:
providing a plurality of sensors; and
averaging the signals from the sensors to compensate for off-axis loads.

18. The method of claim 14, including the step of rigidly coupling the handle of the probe to a load cell including a plurality of strain gages.

19. The method of claim 14, including the step of wirelessly transmitting the pressure measurement to a remote computer display.

20. The method of claim 14, including the step of displaying a blood pressurement in mm/Hg.
